(19) United States
(12) Patent Application Publication
(10) Pub. No.: US 2009/0305945 A1
(43) Pub. Date: Dec. 10, 2009

(54) NOVEL NUTRACEUTICAL COMPOSITIONS

(76) Inventors: Swen Wolfram, Waldshut-Tiengen (DE); Lucas Johannes Cornelis, Maastricht (NL)

Correspondence Address: NIXON & VANDERHYE, PC, 901 NORTH GLEBE ROAD, 11TH FLOOR, ARLINGTON, VA, 22203, US

(21) Appl. No.: 12/296,488

(22) PCT Filed: Apr. 12, 2007
(86) PCT No.: PCT/EP2007/053559

§ 371 Date: Oct. 8, 2008

(30) Foreign Application Priority Data
Apr. 12, 2006 (EP) 06112563.9

(51) Int. Cl. A61K 38/02; A61P 3/10; A61P 3/04

(52) U.S. Cl. 514/2

(57) ABSTRACT

The present invention describes a composition which comprises a protein hydrolysate or a protein hydrolysate and one amino acid hydrolysate for the long term treatment or prevention of type 2 diabetes or pre-diabetes or metabolic syndrome or obesity.

Diabetes mellitus is a widespread chronic disease that hitherto has no cure. The incidence and prevalence of diabetes mellitus is increasing exponentially and it is among the most common metabolic disorders in developed and developing countries. Diabetes mellitus is a complex disease derived from multiple causative factors and characterized by impaired carbohydrate, protein and fat metabolism associated with a deficiency in insulin secretion and/or insulin resistance. This results in elevated fasting and postprandial serum glucose concentrations that lead to complications if left untreated. There are two major categories of the disease, insulin-dependent diabetes mellitus (IDDM, T1DM) and non-insulin-dependent diabetes mellitus (NIDDM, T2DM). T1DM=type 1 diabetes mellitus. T2DM=type 2 diabetes mellitus.

T1DM and T2DM diabetes are associated with hyperglycemia, hypercholesterolemia and hyperlipidemia. The absolute insulin deficiency and insensitivity to insulin in T1DM and T2DM, respectively, leads to a decrease in glucose utilization by the liver, muscle and the adipose tissue and to an increase in the blood glucose levels. Uncontrolled hyperglycemia is associated with increased and premature mortality due to an increased risk for microvascular and macrovascular diseases, including nephropathy, neuropathy, retinopathy, hypertension, stroke, and heart disease. Recent evidence showed that tight glycemic control is a major factor in the prevention of these complications in both T1DM and T2DM. Therefore, optimal glycemic control by drugs or therapeutic regimens is an important approach for the treatment of diabetes.

Long-term complications of patients with T2DM include cardiovascular disease, blindness, neuronal damage, renal failure and diabetic foot disease. A comprehensive overview about the long-term complications of T2DM is provided by the Center for Disease Control and Prevention (http://www.cdc.gov/diabetes/statistics/index.htm accessed on Mar. 11, 2006) and therefore, the most common complications are only briefly mentioned here:

Cardiovascular disease is the leading cause of death in subjects with diabetes, 70-80% of whom will eventually die from it.

73% of diabetics either have a blood pressure>130/80 mmHg or use anti-hypertensive medication.

Diabetes is the leading cause of blindness in adults.

As much as 50% of kidney failure cases are due to diabetes.

More than 60% of subjects with diabetes display signs of neuropathy such as impaired sensation in the feet.

The majority of non-traumatic lower limp amputations occur in diabetic subjects.

Almost one third of people with diabetes have severe periodontal disease.

Poorly controlled diabetes results in an increased rate of spontaneous abortions and can cause major birth defects.

Diabetes during pregnancy can also result in excessively large babies, posing a risk for both mother and child.

Other complications include diabetic ketoacidosis and hyperosmolar coma, which are acute life-threatening events.

Finally, diabetes is also associated with an increased likelihood to acquire other diseases followed by increased mortality from those diseases, which include for example pneumonia and influenza.

Long-term glycemic control is determined by measuring glycosylated hemoglobin (HbA1c) levels, In subjects with chronically high blood glucose levels, the percentage of glycosylated hemoglobin is increased compared to subjects with normal blood glucose levels. The HbA1c concentration can be measured by two reference methods, mass spectroscopy and capillary electrophoresis. A reduction of HbA1c levels in subjects with diabetes suggests that the anti-diabetic therapy or treatment program was successful, e.g. enhanced glycemic control, during the previous 3 months.

Therapy of T2DM initially involves dietary and lifestyle changes, when these measures fail to maintain adequate glycemic control the patients are treated with oral hypoglycemic agents and/or exogenous insulin. The current oral pharmacological agents for the treatment of T2DM include those that increase insulin secretion (sulphonylurea agents), those that improve the action of insulin in the liver (biguanide agents), insulin-sensitizing agents (thiazolidinediones) and agents which act to inhibit the uptake of glucose (α-glucosidase inhibitors). However, currently available agents generally fail to maintain adequate glycemic control in the long term due to progressive deterioration of hyperglycemia, resulting from progressive loss of pancreatic cell function. The proportion of patients able to maintain target glycemia levels decreases markedly over time necessitating the administration of additional/alternative pharmacological agents. Furthermore, the drugs may have unwanted side effects and are associated with high primary and secondary failure rates. Finally, the use of hypoglycemic drugs may be effective in controlling blood glucose levels, but may not prevent all the complications of diabetes. Thus, current methods of treatment for all types of diabetes mellitus fail to achieve the ideals of normoglycemia and the prevention of diabetic complications.

Therefore, although the therapies of choice in the treatment of T1DM and T2DM are based essentially on the administration of insulin and of oral hypoglycemic drugs, there is a need for a safe and effective nutritional supplement with minimal side effects for the treatment and prevention of diabetes. Many patients are interested in alternative therapies which could minimize the side effects associated with high-dose of drugs and yield additive clinical benefits. Patients with diabetes mellitus have a special interest in treatment considered as “natural” with mild anti-diabetic effects and without major side effects, which can be used as adjuvant treatment. T2DM is a progressive and chronic disease, which usually is not recognized until significant damage has occurred to the pancreatic cells responsible for producing insulin (β-cells of islets of Langerhans). Therefore, there is an increasing interest in the development of a dietary supplement that may be used to prevent β-cell damage and thus, the progression to overt T2DM in people at risk especially in elderly who are at high risk for developing T2DM. Protection of pancreatic β-cells may be achieved by decreasing blood glucose and/or lipid levels as glucose and lipids exert damaging effects on β-cells. The reduction of blood glucose levels can be achieved via different mechanisms, for example by enhancing insulin sensitivity and/or by reducing hepatic glucose production. The reduction of blood lipid levels can also be achieved via different mechanisms, for example by enhancing lipid oxidation and/or lipid storage. Another possible strategy to protect pancreatic β-cells would be to decrease oxidative stress. Oxidative stress also causes β-cell damage with subsequent loss of insulin secretion and progression to overt T2DM.

Therefore, T2DM is a complicated disease resulting from coexisting defects at multiple organ sites: resistance to insulin action in muscle and adipose tissues, defective pancreatic insulin secretion, unrestrained hepatic glucose production. Those defects are often associated with lipid abnormalities and endothelial dysfunction. Given the multiple pathophysiological lesions in T2DM, combination therapy is an attractive approach to its management.

The present invention relates to the use of a composition comprising a protein hydrolysate for the long term treatment or prevention of type 2 diabetes or pre-diabetes or metabolic syndrome or obesity or to prevent long-term complications in subjects with type 2 diabetes or pre-diabetes or metabolic syndrome or obesity. Preferably the present invention relates to the use of a composition comprising a protein hydrolysate to decrease 24-hour blood glucose levels in subjects with type 2 diabetes or pre-diabetes or metabolic syndrome or obesity, to increase 24-hour insulin secretion in subjects with type 2 diabetes or pre-diabetes or metabolic syndrome or obesity, to decrease glycosylated hemoglobin concentration (HbA1c) in subjects with type 2 diabetes or pre-diabetes or metabolic syndrome or obesity, to reduce the length of hyperglycaemic periods for subjects with type 2 diabetes or pre-diabetes or metabolic syndrome or obesity, to reduce mortality in subjects with type 2 diabetes or pre-diabetes or metabolic syndrome or obesity, or to prevent long-term complications in subjects with type 2 diabetes or pre-diabetes or metabolic syndrome or obesity.

The present invention relates to the use of a composition comprising a protein hydrolysate as a nutraceutical, preferably a medicament, for the long term treatment or prevention of type 2 diabetes or pre-diabetes or metabolic syndrome or obesity, or to prevent long-term complications in subjects with type 2 diabetes or pre-diabetes or metabolic syndrome or obesity. Preferably the present invention relates to the use of a composition comprising a protein hydrolysate as a nutraceutical, preferably a medicament, to decrease 24-hour blood glucose levels in subjects with type 2 diabetes or pre-diabetes or metabolic syndrome or obesity, to increase 24-hour insulin secretion in subjects with type 2 diabetes or pre-diabetes or metabolic syndrome or obesity, to decrease glycosylated hemoglobin concentration (HbA1c) in subjects with type 2 diabetes or pre-diabetes or metabolic syndrome or obesity, to reduce the length of hyperglycaemic periods for subjects with type 2 diabetes or pre-diabetes or metabolic syndrome or obesity, to reduce mortality in subjects with type 2 diabetes or pre-diabetes or metabolic syndrome or obesity, or to prevent long-term complications in subjects with type 2 diabetes or pre-diabetes or metabolic syndrome or obesity.

The present invention relates to the use of a composition comprising a protein hydrolysate for the manufacture of a nutraceutical, preferably a medicament, for the long term treatment or prevention of type 2 diabetes or pre-diabetes or metabolic syndrome or obesity, or to prevent long-term complications in subjects with type 2 diabetes or pre-diabetes or metabolic syndrome or obesity. Preferably the present invention relates to the use of a composition comprising a protein hydrolysate for the manufacture of a nutraceutical, preferably a medicament, to decrease 24-hour blood glucose levels in subjects with type 2 diabetes or pre-diabetes or metabolic syndrome or obesity, to increase 24-hour insulin secretion in subjects with type 2 diabetes or pre-diabetes or metabolic syndrome or obesity, to decrease glycosylated hemoglobin concentration (HbA1c) in subjects with type 2 diabetes or pre-diabetes or metabolic syndrome or obesity, to reduce the length of hyperglycaemic periods for subjects with type 2 diabetes or pre-diabetes or metabolic syndrome or obesity, to reduce mortality in subjects with type 2 diabetes or pre-diabetes or metabolic syndrome or obesity, or to prevent long-term complications in subjects with type 2 diabetes or pre-diabetes or metabolic syndrome or obesity.

Preferably, the composition which comprises hydrolysate also comprises leucine, preferably at least 70 wt % of the amino acids present in the composition is leucine and that less than 30 wt %, preferably less than 20 wt %, more preferably less than 10 wt % of other amino acids.

In general the protein hydrolysates are administered in an amount sufficient to administer to a subject a daily dosage of 0.01 g per kg body weight to about 3 g per kg body weight, in general the leucine is administered in an amount sufficient to administer to a subject a daily dosage of 0.005 g per kg body weight to about 1 g per kg body weight.

The present invention relates to novel nutraceutical compositions comprising protein hydrolysates or protein hydrolysates and leucine for the treatment or prevention of diabetes mellitus, or other conditions associated with impaired glucose tolerance such as metabolic syndrome and obesity. In another aspect the present invention relates to the use of such compositions as a nutritional supplement for the said treatment or prevention, e.g., as an additive to a multi-vitamin preparations comprising vitamins and minerals which are essential for the maintenance of normal metabolic function but are not synthesized in the body. In still another aspect, the invention relates to a method for the treatment of both type 1 and 2 diabetes mellitus and for the prevention of T2DM in those individuals with pre-diabetes, or impaired glucose tolerance (IGT), or obesity, or metabolic syndrome which comprises administering a protein hydrolysate or a protein hydrolysate and leucine to a subject in need of such treatment.

The compositions of the present invention are particularly intended for the treatment of both T1DM and T2DM and for the prevention of T2DM in those individuals with pre-diabetes, or impaired glucose tolerance (IGT), or metabolic syndrome, or obesity.

The present invention relates to a composition which comprises a protein hydrolysate or a protein hydrolysate and one (free) amino acid. Preferably the one amino acid is leucine. By one amino acid or one amino acid being leucine is understood herein that of the amino acids present in the composition or in the ingredients which are intended for use according to the present invention, that at least 70 wt % of the amino acids present is one amino acid (such as leucine) and than less than 30 wt %, preferably less than 20 wt %, more preferably less than 10 wt % of other amino acids are present. The protein hydrolysate or the combination of a protein hydrolysate and one amino acid, preferably leucine, is advantageously used to decrease 24-hour blood glucose concentrations, preferably for type 2 diabetes or pre-diabetes.

Surprisingly, it is found that the protein hydrolysate or the protein hydrolysate combined with one amino acid can be used for type 2 diabetes or prediabetes, preferably to lower 24-hour glucose concentrations or to reduce the length of hyperglycemic periods or to decrease HbA1c levels.

The compositions comprising a combination of active ingredients, i.e. protein hydrolysate and leucine, synergistically reduce 24-hour glucose levels or the length of hyperglycemic periods or HbA1c levels.

The term nutraceutical as used herein denotes the usefulness in both the nutritional and pharmaceutical field of application. Thus, the novel nutraceutical compositions can find use as supplement to food and beverages, and as pharmaceutical formulations for enteral or parenteral applications, which may be solid formulations such as capsules or tablets, or liquid formulations, such as solutions or suspensions. As will be evident from the foregoing, the term nutraceutical composition also comprises food and beverages containing a protein hydrolysate or a protein hydrolysate and leucine as well as supplement compositions containing the aforesaid active ingredients.

By protein hydrolysate, hydrolysate or hydrolysed protein is meant the product that is formed by enzymatic hydrolysis of the protein, an enriched hydrolysate being a fraction of the protein hydrolysate for example enriched in selected peptides or wherein peptides or polypeptides have been removed from the hydrolysate. So an enriched hydrolysate is preferably a mixture of peptides (or a peptide mixture). The peptide mixture of the invention is therefore a mixture of at least two, preferably at least three, more preferably at least four tryptophane containing peptides. More preferably the mixture comprises a peptide population of which more than 50%, preferably even more than 60%, and most preferably more than 75% of the peptides present have a molecular weight below 500 Da. The protein hydrolysate used in the present invention has a DH of between 7 and 50, preferably a DH of between 10 and 40 and most preferably between 15 and 30.

A “peptide” or “oligopeptide” is defined herein as a chain of at least two amino acids that are linked through peptide bonds. The terms “peptide” and “oligopeptide” are considered synonymous (as is commonly recognized) and each term can be used interchangeably as the context requires. A “polypeptide” is defined herein as a chain containing more than 30 amino acid residues. All (oligo)peptide and polypeptide formulas or sequences herein are written from left to right in the direction from amino-terminus to carboxy-terminus, in accordance with common practice. A protein is defined as used herein as the non-hydrolyzed whey and casein protein. Moreover, protein can also mean hydrolyzed protein. By amino acid is generally meant free amino acid, which is thus not part of a peptide, polypeptide or protein.

A protein hydrolysate can be prepared by incubating a protein source with a single protease or a combination of proteases. Such proteases may be any type of protease including but not limited to endo-proteases, amino peptidases, carboxypeptidases or di- and tri-aminopeptidases.

The protein source can in principle be any protein source. A preferred source is casein or whey protein. A composition comprising whey protein according to the invention may be any composition comprising whey protein such as milk, cream and cheese whey. Whey protein preparations are commercially available in several forms such as whey protein concentrates (WPC) and whey protein isolates (WPI). Suitable protein substrates for hydrolysis also include whole milk, skimmed milk, acid casein, rennet casein, acid whey products or cheese whey products. Moreover, vegetable substrates like wheat gluten, milled barley and protein fractions obtained from, for example, soy, rice or corn are suitable substrates.

A protein hydrolysate can be prepared by contacting the protein substrate with one proteolytic enzyme or a combination of proteolytic enzymes. In case more than one protease is used, these proteases can be added to the protein substrate simultaneously. Alternatively, the proteases can be added to the protein in a predefined sequence. Optionally, the addition of the next protease is preceded by an inactivation of the protease or proteases that were used earlier in the hydrolysis process. Such inactivation may be achieved in various ways and the method of choice depends on the protease that has to be inactivated. Inactivation treatments include but are not limited to heat treatment and a change in pH. Alternatively, commercially available hydrolysates can be used.

The degree of hydrolysis (DH) of a protein substrate is an important parameter. The DH that can be achieved for protein hydrolysate and depends on a large number of parameters, which include but are not limited to the choice for a particular protease, the time that is allowed for the hydrolysis to proceed, the reaction conditions (pH, temperature, salt concentration etc) and the pre-treatment of the protein substrate before it is subjected to hydrolysis by the protease. The DH of the hydrolysate suitable for the process according to the invention may range form 5-50, preferably from 10-40, more preferably form 15-35. The hydrolysate may contain free amino acids. Methods to determine the DH are known to the experts in the field, e.g. the OPA-method described by Church et al. (Anal Biochem (1985) 146, 343). The degree of hydrolysis is the extent to which peptide bonds are broken by the enzymatic hydrolysis reaction.

The hydrolysates can be further processed in various ways, methods including but not limited to spray drying, ultrafiltration, freeze drying, vacuum drying. After drying, the dry material may be grinded and/or sieved in order to obtain fractions of a particular particle size range. Compounds may be added to the hydrolysate to facilitate drying or to influence the final characteristics of the dried hydrolysate such as its tendency to form lumps or its wettability.

In accordance with the present invention it has surprisingly been found that a composition which comprises a protein hydrolysate or a protein hydrolysate and leucine lower 24-hour glucose concentrations or to reduce the length of hyperglycemic periods or to decrease HbA1c levels. Therefore, compositions comprising a protein hydrolysate or a protein hydrolysate and leucine used to prevent or treat both T1DM and T2DM, and for the prevention of T2DM in those individuals with pre-diabetes, impaired glucose tolerance (IGT), or metabolic syndrome, or obesity.

The compositions identified above have been conceived because of their novel action. Owing to the sustained effects of the compositions, not only glycemic control is improved, but in some settings drug dosing can be decreased and adverse effects can be minimized. Thus, although the therapies of choice in the therapeutic treatment of T1DM and T2DM are based essentially on the administration of insulin and of oral hypoglycemic drugs, appropriate nutritional therapy is also of major importance for the successful treatment of diabetics.

A multi-vitamin and mineral supplement may be added to the nutraceutical compositions of the present invention to obtain an adequate amount of an essential nutrients missing in some diets. The multi-vitamin and mineral supplement may also be useful for disease prevention and protection against nutritional losses and deficiencies due to lifestyle patterns and common inadequate dietary patterns sometimes observed in diabetes. Moreover, oxidant stress has been implicated in the development of insulin resistance. Reactive oxygen species may impair insulin stimulated glucose uptake by disturbing the insulin receptor signaling cascade. The control of oxidant stress with antioxidants such as α-tocopherol (vitamin E) ascorbic acid (vitamin C) may be of value in the treatment of diabetes. Therefore, the intake of a multi-vitamin supplement may be added to the above mentioned active substances to maintain a well balanced nutrition.

In a preferred aspect of the invention, the nutraceutical composition of the present invention contains a protein hydrolysate or a protein hydrolysate and leucine. Leucine suitably is present in the composition according to the invention in an amount to provide a daily dosage from about 0.001 g per kg body weight to about 1 g per kg body weight of the subject to which it is to be administered. A food or beverage suitably contains about 0.05 g per serving to about 50 g per serving of leucine. If the nutraceutical composition is a pharmaceutical formulation such formulation may contain leucine in an amount from about 0.001 g to about 1 g per dosage unit, e.g., per capsule or tablet, or from about 0.035 g per daily dose to about 70 g per daily dose of a liquid formulation. Protein hydrolysates suitably are present in the composition according to the invention in an amount to provide a daily dosage from about 0.01 g per kg body weight to about 3 g per kg body weight of the subject to which it is to be administered. A food or beverage suitably contains about 0.1 g per serving to about 100 g per serving of protein hydrolysates. If the nutraceutical composition is a pharmaceutical formulation such formulation may contain protein hydrolysates in an amount from about 0.01 g to about 5 g per dosage unit, e.g., per capsule or tablet, or from about 0.7 g per daily dose to about 210 g per daily dose of a liquid formulation.

Preferred nutraceutical compositions of the present invention comprise a protein hydrolysate or a protein hydrolysate and leucine

### Dosage Ranges (for a 70 Kg Person)

Protein hydrolysates: 0.07-210 g/day
Leucine: 0.005-70 g/day

The following Examples illustrate the invention further.

Pharmaceutical compositions may be prepared by conventional formulation procedures using the ingredients specified below:

### EXAMPLES

### Example 1

### Soft Gelatin Capsule

Soft gelatin capsules are prepared by conventional procedures using ingredients specified below:

Active ingredients. Protein hydrolysate 0.3 g, leucine 0.1 g
Other ingredients: glycerol, water, gelatin, vegetable oil

### Example 2

### Hard Gelatin Capsule

Hard gelatin capsules are prepared by conventional procedures using ingredients specified below:

Active ingredients: Protein hydrolysate 0.7 g, leucine 0.3 g
Other ingredients:
Fillers: lactose or cellulose or cellulose derivatives q.s
Lubricant: magnesium stearate if necessary (0.5%)

### Example 3

### Tablet

Tablets are prepared by conventional procedures using ingredients specified below:

Active ingredients: Protein hydrolysate 0.8 g
Other ingredients: microcrystalline cellulose, silicone dioxide (SiO2), magnesium stearate, crosscarmellose sodium.

B. Food items may be prepared by conventional procedures using ingredients specified below:

### Example 4

### Soft Drink with 30% Juice

Typical serving: 240 ml
Active ingredient:
Protein hydrolysate is incorporated in this food item:
Protein hydrolysates: 1.5-15 g/per serving

A Soft Drink Compound is prepared from the following ingredients:

### I. Juice Concentrates and Water Soluble Flavors

[g]

1.1 Orange concentrate

| | |
|---|---|
| 60.3° Brix, 5.15% acidity | 657.99 |

Lemon concentrate

| | |
|---|---|
| 43.5° Brix, 32.7% acidity | 95.96 |
| Orange flavor, water soluble | 13.43 |
| Apricot flavor, water soluble | 6.71 |
| Water | 26.46 |

1.2 Color

| | |
|---|---|
| β-Carotene 10% CWS | 0.89 |
| Water | 67.65 |

1.3 Acid and Antioxidant

| | |
|---|---|
| Ascorbic acid | 4.11 |
| Citric acid anhydrous | 0.69 |
| Water | 43.18 |

1.4 Stabilizers

| | |
|---|---|
| Pectin | 0.20 |
| Sodium benzoate | 2.74 |
| Water | 65.60 |

1.5 Oil soluble flavors

| | |
|---|---|
| Orange flavor, oil soluble | 0.34 |
| Orange oil distilled | 0.34 |

1.6 Active Ingredients

Active ingredient (this means the active ingredient mentioned above: protein hydrolysate) in the concentrations mentioned above.

Fruit juice concentrates and water soluble flavors are mixed without incorporation of air. The color is dissolved in deionized water. Ascorbic acid and citric acid is dissolved in water. Sodium benzoate is dissolved in water. The pectin is added under stirring and dissolved while boiling. The solution is cooled down. Orange oil and oil soluble flavors are premixed. The active ingredients as mentioned under 1.6 are dry mixed and then stirred preferably into the fruit juice concentrate mixture (1.1).

In order to prepare the soft drink compound all parts 3.1.1 to 3.1.6 are mixed together before homogenizing using a Turrax and then a high-pressure homogenizer (p₁=200 bar, P₂=50 bar).

II. A Bottling Syrup is Prepared from the Following Ingredients:

| | [g] |
|---|---|
| Softdrink compound | 74.50 |
| Water | 50.00 |
| Sugar syrup 60° Brix | 150.00 |

The ingredients of the bottling syrup are mixed together. The bottling syrup is diluted with water to 1 l of ready to drink beverage.

Variations:

Instead of using sodium benzoate, the beverage may be pasteurized. The beverage may also be carbonized.

### Example 5

### Five Cereal Bread

Typical serving: 50 g
Active ingredient:
Protein hydrolysate is incorporated in this food item:
Protein hydrolysate: 1.5-15 g/per serving

| Other components: | [%] |
|---|---|
| Five cereal flour | 56.8 |
| Water | 39.8 |
| Yeast | 2.3 |
| Salt | 1.1 |

The yeast is dissolved in a part of the water. All ingredients are mixed together to form a dough. Salt is added at the end of the kneading time. After fermentation, the dough is reworked and divided before a loaf is formed. Before baking, the surface of the loaf is brushed with water and sprinkled with flour.

Procedure:

| Kneading: | | |
|---|---|---|
| Spiral kneading system | 4 min 1^{st} gear 5 min 2^{nd} gear | |
| Dough proofing: | 60 | min |
| Dough temperature: | 22-24° | C. |
| Proofing time: | 30 | min |
| Baking: | | |
| Oven: | Dutch type oven | |
| Baking temperature: | 250/220° | C. |
| Baking time: | 50-60 | min |

### Example 6

### Cookies Type Milano

Typical serving: 30 g
Active ingredients:
Protein hydrolysates and leucine are incorporated in this food item:
Protein hydrolysates: 0.9-9 g/per serving
Leucine: 0.3-3 g/per serving

| Other components: | [g] |
|---|---|
| Wheat Flour, type 550 | 41.0 |
| Sugar | 20.5 |
| Fat/Butter | 20.5 |
| Whole egg (liquid) | 18.0 |
| Lemon Flavor | q.s. |
| Baking agent | q.s. |

All ingredients are added slowly under mixing to form a sweet short pastry.

Afterwards, the pastry is kept cool (4° C.) for at least 2 hours before flattening the pastry to a thickness of approx. 5 mm. Pieces are cut out and brushed with egg yolk on the surface before baking.

Baking:

Oven: fan oven
Baking temperature: 180° C.
Baking time: 15 min

### Example 7

### Toast

Typical serving: 100 g
Active ingredients:
Protein hydrolysate and leucine are incorporated in this food item:
Protein hydrolysate: 1.8-18 g/per serving
Leucine: 0.6-6 g/per serving

| Other components: | [%] |
|---|---|
| Wheat Flour, type 550 | 55.4 |
| Water | 33.2 |
| Yeast | 2.8 |
| Salt | 1.1 |
| Fat/Butter | 5.5 |
| Malt | 0.6 |
| Emulsifier baking agent | 1.4 |

The yeast is dissolved in a part of the water. All ingredients are mixed together to form a dough. Salt is added at the end of the kneading time. Afterwards, the dough is reworked, divided and placed in a baking tin for fermentation. After baking, the loaf is unmoulded directly.

Procedure:

Kneading:

Spiral kneading system 5-6 min 1^{st} gear; 3-4 min 2^{nd} gear
Dough proofing: none
Dough temperature: 22-24° C.
Proofing time: 40 min

Baking:

Oven: Dutch type oven
Baking temperature: 220° C.
Baking time: 35-40 min

### Example 8

### Yoghurt

### Set Type; 3.5% Fat

Typical serving: 225 g
Active ingredients:
Protein hydrolysate is incorporated in this food item:
Protein hydrolysate: 1.5-15 g/per serving

| Other components: | [%] |
|---|---|
| Full fat milk (3.8% fat) | 90.5 |
| Skimmed milk powder | 2.0 |
| Sugar | 5.0 |
| Culture | 2.5 |

The milk is heated to 350° C. before addition of milk powder, stabilizer, sugar and active ingredients. This mixture is heated to 65° C. to dissolve all ingredients. Then the mixture is homogenized in a high-pressure homogenizer (p₁=150 bar, P₂=50 bar) at 65° C. This emulsion is then pasteurized at 80° C. for 20 minutes. After cooling to 45° C. natural yoghurt/culture is added and mixed. Then this mixture is filled into cups and fermented at 45° C. for 3-4 hours until a pH of 4.3 is reached and then stored at 4° C.

### Example 9

### Yoghurt

### Stirred Type; 3.5% Fat

Typical serving: 225 g
Protein hydrolysate is incorporated in this food item:
Protein hydrolysates: 0.5-5 g/per serving

| Other components: | [%] |
|---|---|
| Full fat milk (3.8% fat) | 90.2 |
| Skimmed milk powder | 2.0 |
| Stabilizer | 0.3 |
| Sugar | 5.0 |
| Culture | 2.5 |

The milk is heated to 35° C. before addition of milk powder, stabilizer, sugar and active ingredients. This mixture is heated to 65° C. to dissolve all ingredients before homogenization in a high-pressure homogenizer (p₁=150 bar, P₂=50 bar) at 65° C. This emulsion is then pasteurized at 80° C. for 20 minutes. After cooling to 45° C. natural yoghurt/culture is added and mixed, followed by fermentation at 450° C. for 3-4 hours until a pH of 4.3 is reached. After cooling and stirring vigorously, the yoghurt is filled in cups and stored at 4° C.

### Example 10

### Ice Cream

### 8% Fat

typical serving: 85 g
Active ingredients:
Protein hydrolysate and leucine are incorporated in this food item:
Protein hydrolysates: 0.3-3 g/per serving
Leucine: 0.1-1 g/per serving

| Other components: | [g] |
|---|---|
| Milk (3.7% fat) | 600.00 |
| Cream (35% fat) | 166.00 |
| Skim milk powder | 49.10 |
| Sugar | 109.00 |
| Glucose syrup 80% | 70.00 |
| ice cream stabilizer | 5.00 |
| Flavor | q.s. |
| Color | q.s |

Sugar, skim milk powder and stabilizer are added to the milk and cream, mixed and heated to 45° C. Then the color as stock solution and the glucose syrup is added as well as the active ingredients. The mix is heated up and pasteurized (20 min, 80° C.). Then a homogenization step takes place. Afterwards the mix is cooled down under constant stirring and the flavor is added at 5° C. The mix maturated at 5° C. during at least 4 h and then passed through an ice cream machine (overrun ca. 100%). The ice cream is filled into cups and stored at −20 to −30° C.

### Example 11

### Wine Gums

Active ingredient:
Protein hydrolysate is incorporated in this food item:
Protein hydrolysate: 0.15-1.5 g/per 30 g

| Other components: | [g] |
|---|---|
| Gelatin 200 Bloom | 80.0 |
| Water I | 125.0 |
| Sugar crys. | 290.0 |
| Water II | 120.0 |
| Glucose-syrup DE 38 (carbohydrate source) | 390.0 |
| Citric acid | 10.0 |
| Flavor | 2.0 |
| Color | q.s. |
| Yield ca | 1000.0 |

Disperse gelatin in water I, stir and dissolve by heating over a stream bath or using a microwave. Mix sugar with water II and bring to boiling until a clear solution is obtained. Remove from heat source. Mix with glucose syrup while dissolved sugar solution is still hot. Slowly add the gelatin solution. Let rest until foam on surface can be removed and 60-65° C. is reached. Add flavor, citric acid and the color solution as well as active ingredients under stirring. Deposit into moulds printed into starch trays and let sit for at least 48 hours at RT. Remove starch powder and polish with oil or wax. Dry at RT and package into airtight pouches

### Example 12

This example shows the effects of consumption of a drink containing a protein hydrolysate and leucine together with a mixed meal. The drink containing a protein hydrolysate and leucine or a placebo-drink without the protein hydrolysate and leucine was consumed 3 times during the day together with breakfast, lunch, and dinner. Eleven male subjects with long-term T2DM participated in 2 trials, in which a 24-hour blood glucose profile was determined.

To determine the 24-hour blood glucose profile a microdialysis fiber (Medica, Medolla, Italy) was inserted in the peri-umbilical region. The micro-fiber was subsequently connected to a portable continuous glucose-measuring device (CGMS; GlucoDay®, A. Menarini Diagnostics, Firenze, Italy). Thereafter, subjects were provided with their diet and were allowed to return home and resume their normal daily activities. The following day the subjects consumed their designated meals, drinks and snacks at set time-points. After each main meal (i.e. breakfast, lunch and dinner) the subjects drank a bolus beverage (4 mL/kg) containing either the protein hydrolysate/leucine mixture (PRO) or flavored water (PLA). The subsequent day, subjects reported back to the laboratory where the CGMS was removed. CGMS data of the second day (from 0700 to 0700) were used for data analyses. The CGMS is an ambulant continuous glucose monitoring system based on the microdialysis technique and allows continuous glucose monitoring for a period of 48 h (13). A microdialysis fiber (Medica, Medolla, Italy) with an internal diameter of 0.17 mm and a cut-off weight of 18 kD was inserted in the peri-umbilical region, without anesthesia, using an 18-gauge Teflon catheter as a guide [Meyerhoff et al. Diabetologia (1992) 35, 1087]. The micro-fiber was subsequently connected to the portable continuous glucose-measuring device (GlucoDay®, A. Menarini Diagnostics, Firenze, Italy). The device consists of a peristaltic pump that pumps Dulbecco's solution at a rate of 10 μL-min through the microdialysis fiber. The subcutaneous interstitial fluid is taken up by the microdialysis fiber and is transported to the measuring cell. The glucose sensor, consisting of immobilized glucose oxidase measures the glucose concentration every minute and stores an average value every 3 min up until a 48 h period. The entire device, including the perfusion solution and the waste-bag, weighs about 250 g and is worn in a pouch under the subjects' clothes. The acquired data were downloaded from the device to a personal computer with GlucoDay® software (V3.0.5). Values reported by the CGMS were converted into glucose values using the capillary glucose measurements as calibration values. The efficacy and the accuracy of the CGMS device has been validated for both diabetic [Maran et al. Diabetes Care (2002) 25, 347; Wentholt et al. Diabetes Care (2005) 28, 2871] and non-diabetic subjects [Maran et al. Diabetes Metab Res Rev (2004) 20, S50; Poscia et al. Biosens Bioelectron (2003) 18, 891; Varalii et al. Biosens Bioelectron (2003) 18, 899]. To quantify and compare the prevalence of hyperglycemia between groups and trials, the amount of time during which glucose concentrations were above 10 mmol/L was calculated.

The 24-hour blood glucose responses were 5.2±0.25 mol/24 h/l in subjects consuming the placebo drink and 4.6±0.3 mol/24 h/l in subjects consuming the drink containing a protein hydrolysate and leucine (P<0.05). This means that the consumption of 3 drinks containing a protein hydrolysate and leucine during the day resulted in a reduction of 24-hour blood glucose by 11.2%. It is an important goal for subjects with T2DM to reduce blood glucose levels during the day. The American Diabetes Association defined a blood glucose concentration below 10 mmol/l at random measurements throughout the day as one of the main goals of for subjects with T2DM. Random blood glucose concentrations above 10 mmol/l are known to be associated with increased mortality and more severe long-term complications in subjects with T2DM. If blood glucose concentrations exceed 10 mmol/l damage to many organs such as kidney, retina, and blood vessels occur. The longer blood glucose concentrations are above 10 mmol/l (hyperglycemic period) the greater is the damage exerted. Surprisingly, the consumption of a drink containing a protein hydrolysate and leucine resulted in a pronounced reduction in the length of hyperglycemic periods of 26% when compared to consumption of the placebo drink (see also table 1). We conclude that consumption of a protein hydrolysate and leucine significantly reduces 24-hour blood glucose concentrations and the length of the hyperglycemic periods in subjects with T2DM. Therefore, the invention is useful also for the reduction of mortality and for the prevention of long-term complications in subjects with type 2 diabetes or prediabetes or metabolic syndrome or obesity.

Table 1. Hyperglycaemic periods with blood glucose above 10 mmol/l (expressed in hours:minutes) of subjects consuming placebo or 0.3 g/kg protein hydrolysate+0.1 g/kg leucine in a drink. The drink was provided 3 times during the day and was consumed together with a mixed meal (breakfast, lunch, dinner). Postprandial periods after breakfast, lunch and dinner were defined to be 4, 6 and 6 hours, respectively. * significantly different from placebo (P<0.05).

| | Placebo | Protein hydrolysate | Delta % |
|---|---|---|---|
| 24 h | 13:08 | 09:42 * | −26.1 |
| Breakfast | 03:20 | 03:00 * | −10.0 |
| Lunch | 04:26 | 03:39 * | −17.7 |
| Dinner | 02:16 | 02:17 | 0.7 |

1. Use of a composition comprising a protein hydrolysate for the long term treatment or prevention of type 2 diabetes or pre-diabetes or metabolic syndrome or obesity or to prevent long-term complications in subjects with type 2 diabetes or pre-diabetes or metabolic syndrome or obesity, preferably to decrease 24-hour blood glucose levels in subjects with type 2 diabetes or pre-diabetes or metabolic syndrome or obesity, to increase 24-hour insulin secretion in subjects with type 2 diabetes or pre-diabetes or metabolic syndrome or obesity, to decrease glycosylated hemoglobin concentration (HbA1c) in subjects with type 2 diabetes or pre-diabetes or metabolic syndrome or obesity, to reduce the length of hyperglycaemic periods for subjects with type 2 diabetes or pre-diabetes or metabolic syndrome or obesity, to reduce mortality in subjects with type 2 diabetes or pre-diabetes or metabolic syndrome or obesity, or to prevent long-term complications in subjects with type 2 diabetes or pre-diabetes or metabolic syndrome or obesity.

2. Use of a composition comprising a protein hydrolysate as a nutraceutical, preferably a medicament for the long term treatment or prevention of type 2 diabetes or pre-diabetes or metabolic syndrome or obesity, or to prevent long-term complications in subjects with type 2 diabetes or pre-diabetes or metabolic syndrome or obesity, preferably to decrease 24-hour blood glucose levels in subjects with type 2 diabetes or pre-diabetes or metabolic syndrome or obesity, to increase 24-hour insulin secretion in subjects with type 2 diabetes or pre-diabetes or metabolic syndrome or obesity, to decrease glycosylated hemoglobin concentration (HbA1c) in subjects with type 2 diabetes or pre-diabetes or metabolic syndrome or obesity, to reduce the length of hyperglycaemic periods for subjects with type 2 diabetes or pre-diabetes or metabolic syndrome or obesity, to reduce mortality in subjects with type 2 diabetes or pre-diabetes or metabolic syndrome or obesity, or to prevent long-term complications in subjects with type 2 diabetes or pre-diabetes or metabolic syndrome or obesity.

3. Use of a composition comprising a protein hydrolysate for the manufacture of a nutraceutical, preferably a medicament for the long term treatment or prevention of type 2 diabetes or pre-diabetes or metabolic syndrome or obesity, or to prevent long-term complications in subjects with type 2 diabetes or pre-diabetes or metabolic syndrome or obesity, preferably to decrease 24-hour blood glucose levels in subjects with type 2 diabetes or pre-diabetes or metabolic syndrome or obesity, to increase 24-hour insulin secretion in subjects with type 2 diabetes or pre-diabetes or metabolic syndrome or obesity, to decrease glycosylated hemoglobin concentration (HbA1c) in subjects with type 2 diabetes or pre-diabetes or metabolic syndrome or obesity, to reduce the length of hyperglycaemic periods for subjects with type 2 diabetes or pre-diabetes or metabolic syndrome or obesity, to reduce mortality in subjects with type 2 diabetes or pre-diabetes or metabolic syndrome or obesity, or to prevent long-term complications in subjects with type 2 diabetes or pre-diabetes or metabolic syndrome or obesity.

4. Use according to claim 1, wherein the composition comprises leucine.

5. Use of claim 4 wherein at least 70 wt % of the amino acids present in the composition is leucine and that less than 30 wt %, preferably less than 20 wt %, more preferably less than 10 wt % of other amino acids.

6. Use according to claim 1, wherein protein hydrolysates are administered in an amount sufficient to administer to a subject a daily dosage of 0.01 g per kg body weight to about 3 g per kg body weight.

7. Use according to claim 1, wherein leucine is administered in an amount sufficient to administer to a subject a daily dosage of 0.005 g per kg body weight to about 1 g per kg body weight.

8. Use according to claim 1, wherein the hydrolysate or hydrolysate and leucine is used in the form of a food, beverage or a supplement composition for a food or beverage or in an pharmaceutically acceptable dosage form.
